(19) United States
(12) Patent Application Publication
(10) Pub. No.: US 2026/0354914 A1
(43) Pub. Date: Nov. 12, 2026

(54) PLUG-AND-PLAY ARM FOR SPINAL ROBOTICS

(71) Applicant: Medtronic Navigation, Inc., Caesarea, CO (US)

(72) Inventors: Weijun Xu, Shanghai (CN); Wei Tang, Shanghai (CN); Yingying Liu, Shanghai (CN); Wuqian Li, Shanghai (CN)

(21) Appl. No.: 19/125,766

(22) PCT Filed: Nov. 16, 2022
(86) PCT No.: PCT/CN2022/132221

§ 371 Date: Apr. 30, 2025

(51) Int. Cl. A61B 34/32; A61B 34/30

(52) U.S. Cl. CPC A61B 34/32; A61B 2034/303

(57) ABSTRACT

Methods and systems (100) include identifying one or more properties associated with a robotic arm (116). The robotic arm (116) is plug-and-play compatible with a coupling interface of the systems (100). The methods and systems (100) include determining instruction operations and control parameters associated with controlling the robotic arm (116) based on the one or more properties. The methods and systems (100) include controlling the robotic arm (116) in association with a surgical procedure based on the instruction operations and control parameters. The methods and systems (100) include removably coupling the robotic arm (116) to a support structure (154,155), with or without an extension member (140).

### FIELD OF INVENTION

The present disclosure is generally directed to robotic assisted surgery, and relates more particularly to plug-and-play robotic arms for robotic assisted surgery.

### BACKGROUND

Surgical robots may assist a surgeon or other medical provider in carrying out a surgical procedure, or may complete one or more surgical procedures autonomously. Providing controllable linked articulating members allows a surgical robot to reach areas of a patient anatomy during various medical procedures.

### BRIEF SUMMARY

Example aspects of the present disclosure include:

A system including: a processor; and a memory storing instructions thereon that, when executed by the processor, cause the processor to: identify one or more properties associated with a robotic arm, wherein the robotic arm is plug-and-play compatible with a coupling interface of the system; determine one or more instruction operations associated with controlling the robotic arm based on the one or more properties; and control the robotic arm in association with a surgical procedure by transmitting one or more surgical commands based on the one or more instruction operations.

Any of the aspects herein, wherein the instructions are further executable by the processor to: detect a removable coupling established between the robotic arm and the coupling interface, wherein the removable coupling includes at least one of: a mechanical connection established between the robotic arm and the coupling interface; and an electrical connection established between the robotic arm and the coupling interface, wherein identifying the one or more properties associated with the robotic arm is in response to detecting the removable coupling.

Any of the aspects herein, wherein the instructions are further executable by the processor to: retrieve the one or more properties associated with the robotic arm from a memory stored on the robotic arm.

Any of the aspects herein, wherein the one or more properties associated with the robotic arm include at least one of: status information associated with the robotic arm; and identification information associated with the robotic arm.

Any of the aspects herein, wherein the one or more properties associated with the robotic arm include at least one of: a hardware version associated with the robotic arm; and a software version associated with the robotic arm.

Any of the aspects herein, wherein the one or more properties associated with the robotic arm include at least one of: a type associated with the robotic arm; one or more functions associated with the robotic arm; and a range of motion associated with the robotic arm.

Any of the aspects herein, further including: determine one or more control parameters associated with controlling the robotic arm based on the one or more properties associated with the robotic arm, wherein the one or more control parameters include at least one of target pose information of the robotic arm and second target pose information of an end effector of the robotic arm, wherein controlling the robotic arm includes by transmitting the one or more surgical commands based on the one or more control parameters.

Any of the aspects herein, wherein determining the one or more instruction operations, determining the one or more control parameters, or both is based on at least one of: one or more characteristics of a subject associated with the surgical procedure; and one or more characteristics of an anatomical element of the subject in association with the surgical procedure.

Any of the aspects herein, wherein the instructions are further executable by the processor to: identify one or more second properties associated with an extension member, wherein: the extension member is plug-and-play compatible with the coupling interface and the robotic arm; and the extension member is removably coupled to the coupling interface and removably coupled to the robotic arm; determine one or more second control parameters associated with controlling the extension member and the robotic arm based on identifying the one or more second properties; and control the extension member and the robotic arm in association with the procedure by transmitting the one or more surgical commands based on the one or more second control parameters.

Any of the aspects herein, wherein the one or more second properties include at least one of: a type associated with the extension member; one or more functions associated with the extension member; and a range of motion associated with the extension member.

Any of the aspects herein, wherein determining the one or more instruction operations is based on a target objective associated with performing the procedure.

Any of the aspects herein, wherein the instructions are further executable by the processor to: determine pose information of the robotic arm, pose information of an extension member removably coupled to the robotic arm, or both in response to at least one of: receiving data from the robotic arm; and receiving second data from an extension member, wherein the extension member is removably coupled to the coupling interface.

Any of the aspects herein, wherein the coupling interface is associated with one or more support structures of the system.

A system including: a robotic arm; and robot management circuitry that manages control of the robotic arm by: identifying one or more properties associated with the robotic arm, wherein the robotic arm is plug-and-play compatible with a mounting component of the system; determining one or more instruction operations associated with controlling the robotic arm based on identifying the one or more properties; and controlling the robotic arm in association with a surgical procedure by transmitting one or more surgical commands based on the one or more instruction operations.

Any of the aspects herein, wherein the robot management circuitry is to: detect a removable coupling established between the robotic arm and the coupling interface, wherein the removable coupling includes at least one of: a mechanical connection established between the robotic arm and the coupling interface; and an electrical connection established between the robotic arm and the coupling interface, wherein identifying the one or more properties associated with the robotic arm is in response to detecting the removable coupling

Any of the aspects herein, wherein the robot management circuitry further manages control of the robotic arm by: determining one or more control parameters associated with controlling the robotic arm based on identifying the one or more properties associated with the robotic arm, wherein controlling the robotic arm includes by transmitting the one or more surgical commands based on identifying the one or more control parameters.

Any of the aspects herein, wherein the robot management circuitry further manages control of the robotic arm by: identifying one or more second properties associated with an extension member, wherein: the extension member is plug-and-play compatible with the coupling interface and the robotic arm; and the extension member is removably coupled to the coupling interface of and removably coupled to the robotic arm; determining one or more second control parameters associated with controlling the extension member and the robotic arm based on identifying the one or more second properties; and controlling the extension member and the robotic arm in association with the procedure by transmitting the one or more surgical commands based on the one or more second control parameters.

Any of the aspects herein, further including a support structure including the mounting component, wherein: the mounting component includes one or more position-mounting interfaces, one or more power interfaces, and one or more data communication interfaces; and the robotic arm is plug-and-play compatible with the mounting component based on one or more second position-mounting interfaces, one or more second power interfaces, and one or more second data communication interfaces of the robotic arm.

Any of the aspects herein, further including an extension member removably coupled to the robotic arm and to the mounting component, wherein: a first end of the extension member is plug-and-play compatible with the mounting component based on one or more position-mounting interfaces, one or more power interfaces, and one or more data communication interfaces of the extension member; and a second end of the extension member is plug-and-play compatible with the robotic arm based on one or more second position-mounting interfaces, one or more second power interfaces, and one or more second data communication interfaces of the extension member.

A method including: electronically receiving status information associated with a robotic arm of a system, wherein the status information includes an indication that the robotic arm is in an active state, wherein the robotic arm is plug-and-play compatible with a coupling interface of the system; and determining, in response to electronically receiving the status information and based on one or more properties associated with the robotic arm, one or more instruction operations associated with controlling the robotic arm; and controlling the robotic arm in association with a surgical procedure by electronically communicating one or more surgical commands corresponding to the one or more instruction operations to the robotic arm.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIG. 1A is a block diagram of a system according to at least one implementation of the present disclosure. FIGS. 1B and 1C illustrate example implementations of the system in accordance with aspects of the present disclosure.

FIG. 2 illustrates an example process flow in accordance with aspects of the present disclosure.

FIG. 3 illustrates an example process flow in accordance with aspects of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any implementations of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Some robotic systems may support surgical procedures associated with operating on an anatomical element (e.g., the spine, etc.) of a subject. In some cases, a spinal robotic system may include a workstation and a robotic arm controllable by the workstation. The workstation may control the robotic arm by issuing a set of commands. For example, based on the issued commands, the workstation may control the robotic arm to reach a planned position (e.g., coordinates, trajectory, orientation, etc.) and provide a surgeon with an accurate and stable trajectory. Some robotic systems may support robotic arms that can be detached from the workstation and fixed to the side rail of an operating bed. Some robotic systems may support a robotic arm fixed on a cart.

In some robotic systems, the working range of a robotic arm may be limited, which may thereby impact the ease of use of the robotic arm with respect to a surgical procedure. Further, the weight of some robotic arms may negatively impact a user's ability to install a robotic arm to an operating bed. For example, due to the weight of some robotic arms, switching out one robotic arm for another robotic arm to adjust the working range may not be a feasible option for a user. In some other cases, some robotic systems may only support a single type of robotic arm. Such support for only a single type of robotic arm may thereby prevent surgical expansion.

Aspects of the present disclosure described herein support a portable plug-and-play robotic arm. The robotic arm may be separate from a workstation. The robotic arm may include a plug-and-play interface supportive of mounting the robotic arm to a support structure, for example, at a connection interface of the support structure. The support structure may be, for example, an operating table, a ceiling mount structure, a wall mount structure, a support structure of a robot, or the like.

In the example of an operating table, the connection interface of the operating table may be located at any portion (e.g., a side portion, a front portion, etc.) of the operating table. In some aspects, the connection interface of the operating table may be the same as the connection interface of the robotic arm. For example, the connection interface of the operating table and the connection interface of the robotic arm may each include respective configurations (e.g., arrangement, positioning) of a power supply connector, a communication connector, and position mounting holes supportive of removably coupling the robotic arm to the operating table. The connection interfaces may support the transmission of data and power between the operating table (or a computing system associated with the operating table) and the robotic arm.

Aspects of the robotic arm support relatively quick installation to a support structure (e.g., operating table, ceiling mount structure, wall mount structure, support structure of a robot, etc.). For example, the robotic arm may be designed to be lightweight compared to some other robotic arms. In some examples, support structures (e.g., an operating table, etc.) described herein may include multiple connection interfaces compatible with the robotic arm, thereby supporting multiple locations at which the robotic arm may be removably coupled. For example, aspects of the present disclosure support implementations in which a user may relocate the robotic arm from one connection interface to another connection interface as needed with respect to performing a surgical procedure. Such example features extend the working range of the robotic arm.

Example aspects of the robotic arm and the corresponding plug-and-play interface are described herein. The robotic arm may be powered on via the support structure for example, by installing the robotic arm to the support structure. For example, the robotic arm may be in a powered on state once the robotic arm is installed on the support structure (e.g., once the power supply connector of the robotic arm is electrically coupled to the power supply connector of the support structure). In an example, the robotic arm may receive power from the support structure via the power supply connector of the robotic arm.

The workstation may read status data from the robotic arm and implement different algorithms associated with calculating parameters for a surgical procedure. For example, the status data may indicate that the robotic arm is powered on and removably coupled to the support structure. In some cases, the status data may indicate the connection interface of the support structure to which the robotic arm is connected. In some cases, the workstation may read identification information from the robotic arm (e.g., via the connection interface of the robotic arm) and implement algorithms for calculating a screw trajectory for the surgical procedure. In an example, based on the identification information, the workstation may identify a hardware version and/or software version of the robotic arm, based on which the workstation may implement the algorithms.

Aspects of the plug-and-play features of the robotic arm support advantageous implementations in which a single workstation may be compatible with multiple robotic arms for multiple types of surgical procedures (e.g., trauma surgery, spine surgery, joint surgery, etc.). In some aspects, the single workstation may support multiple types of surgical procedures at the same time, using different types of robotic arms. As described herein, the workstation may implement different therapy algorithms based on the identification information read from the connection interface of the robotic arm. The lightweight design of the robotic arm may support relatively quick installation and replacement by a user, thereby supporting implementations in which any combination of robotic arms and robotic arm types may be connected to a support structure (e.g., operating table, etc.) for performing a surgical procedure.

According to example aspects of the present disclosure, a portable plug-and-play extension joint is described that is compatible with the robotic arm described herein. The terms “extension joint,” “extension member,” “extension arm,” and “extension component” may be used interchangeably herein. The extension joint may further extend the working range of a robotic arm. The extension joint may have the same connection interface at respective ends (e.g., two ends) of the extension joint. In an example, a first end which is installed to a support structure (e.g., operating table, ceiling mounting structure, etc.) and powered on will be the “master,” and a second end which is electrically and mechanically connected to the robotic arm will be the “slave.” Aspects of the present disclosure support extension joints of any combination of lengths, any quantity of joints, or the like.

The workstation may read stored data from the extension joint and determine, from the data, pose information of the second end. The first end and the second end of the extension joint may have respective connection interfaces that are compatible with support structures and robotic arms described herein. Example aspects of the connection interfaces of the extension joint, the support structure, and the robotic arm are later described herein.

The extension joint may be powered on via the support structure (e.g., operating table, etc.), for example, by installing the extension joint to the support structure. For example, the extension joint may be in a powered on state once the extension joint is installed on the support structure (e.g., once a power supply connector of the extension joint is electrically coupled to the power supply connector of the support structure). In an example, the extension joint may receive power from the support structure via the power supply connector of the extension joint.

The workstation may read status data from the extension joint and implement different algorithms associated with calculating parameters for a surgical procedure. For example, the status data may indicate that the extension joint is powered on and coupled to the support structure. In some cases, the status data may indicate the connection interface of the support structure to which the extension joint is connected. In some examples, the status data may indicate that a robotic arm is coupled to another end of the extension joint.

In some cases, the workstation may read identification information from the extension joint (e.g., via the connection interface of the extension joint) and implement algorithms for calculating a screw trajectory for the surgical procedure. In an example, based on the identification information, the workstation may identify a hardware version and/or software version of the extension joint, based on which the workstation may implement the algorithms. In the example in which a robotic arm is coupled to the extension joint, the workstation may read identification information described herein with respect to the robotic arm via the extension joint.

Accordingly, for example, aspects of the extension joint support extending the working range of a robotic arm. In an example case, the working range of a robotic arm (e.g., fully extended length of the robotic arm, trajectory of an end effector of the robotic arm based on quantity of joints, etc.) connected to a support structure may be insufficient for performing a surgical procedure. Aspects of the present disclosure support connecting the robotic arm to the support structure via the extension joint may provide an overall working range supportive of performing the surgical procedure. For example, implementations using the extension joint (or multiple extension joints) and the robotic arm may provide a fully extended length and/or additional possible trajectories of the end effector (e.g., due to an increased quantity of joints) supportive of performing the surgical procedure. The extension joint may be mountable to any support structure described herein, for example, an operating table, a ceiling mount support structure, a wall mount support structure, a support structure of a robot device, and the like.

Aspects of the plug-and-play features of the extension joint support advantageous implementations in which a single workstation may be compatible with multiple extension joints and robotic arms for multiple types of surgical procedures (e.g., trauma surgery, spine surgery, joint surgery, etc.). In some aspects, for a case in which a robotic arm of a given type is to be used for a target surgical procedure, the extension joint described herein may supplement the working range of the robotic arm in association with successfully completing the surgical procedure. Accordingly, for example, such implementations may provide cost benefits in that the manufacturing of additional robotic arms of the same type (but different lengths) may be avoided. In a further example, as the extension joint may enable a robotic arm to reach a target location in association with the surgical procedure, a user may avoid having to find a replacement robotic arm of the same type but different length, thereby providing increased user convenience.

The connection interfaces of the extension joint may be compatible with the respective connection interfaces of the support structure and the robotic arms described herein, thereby providing plug-and-play interchangeability and flexibility. Such interchangeability and flexibility may support reduced system setup time. For example, the lightweight design and connection interface compatibility of the robotic arms and extension joints may support relatively quick installation and replacement by a user, thereby supporting implementations in which any combination of extension joints and robotic arms may be connected to a support structure (e.g., operating table, etc.) for performing a surgical procedure.

In some aspects, the lightweight design and flexibility with respect to different mechanical and/or electrical mounting structures (e.g., operating table, a robot, a ceiling-mounted support structure, etc.) support advantageous implementations which may avoid exceeding the weight capacity of an operating table due to the weight of a patient and the weight of the robotic arms. For example, in the case of a relatively heavy patient, aspects of the present disclosure support the removal of a robotic arm from an operating table and attaching the same robotic arm to another support structure (e.g., a ceiling-mounted support structure, a wall-mounted support structure, a robot-mounted support structure, etc.) to meet the weight capacity of the operating table. In some non-limiting example implementations, each of the robotic arms described herein may weigh from about 11 pounds to about 22 pounds, which is relatively lighter in comparison to other surgical robotic arms.

Example aspects of the robotic arm and the extension joint are described with reference to the following Figures.

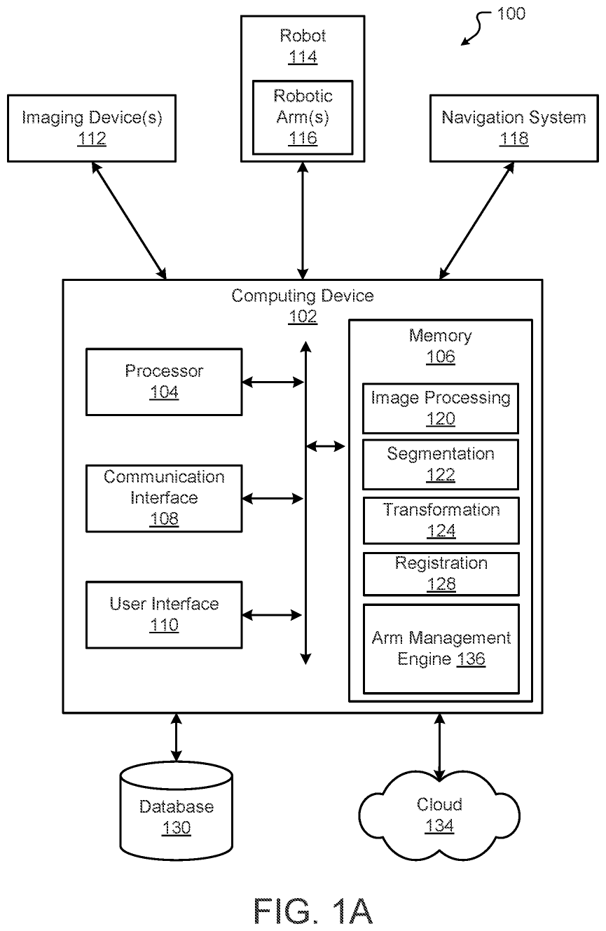

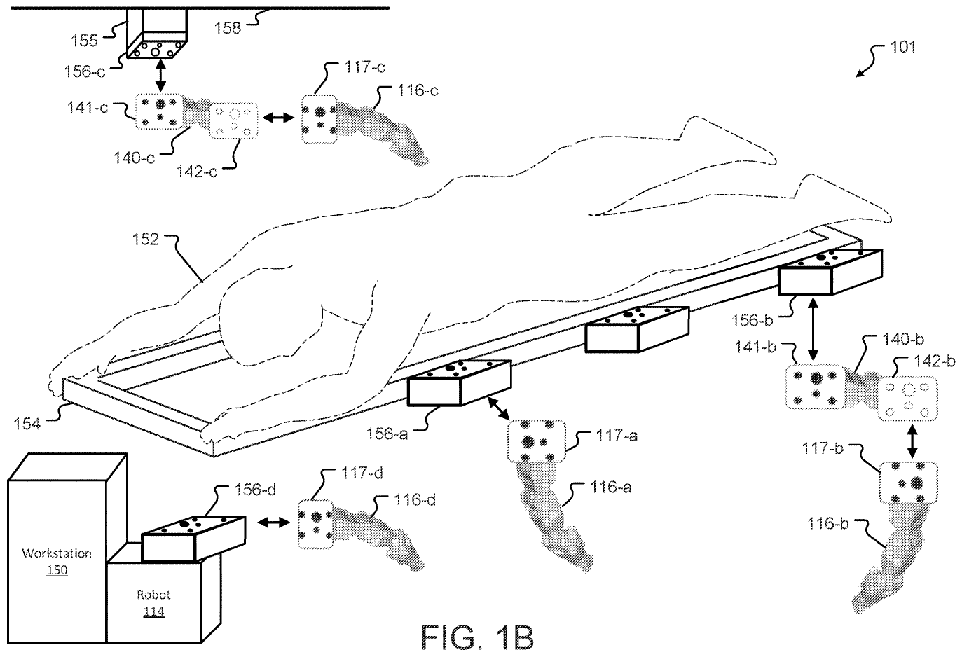

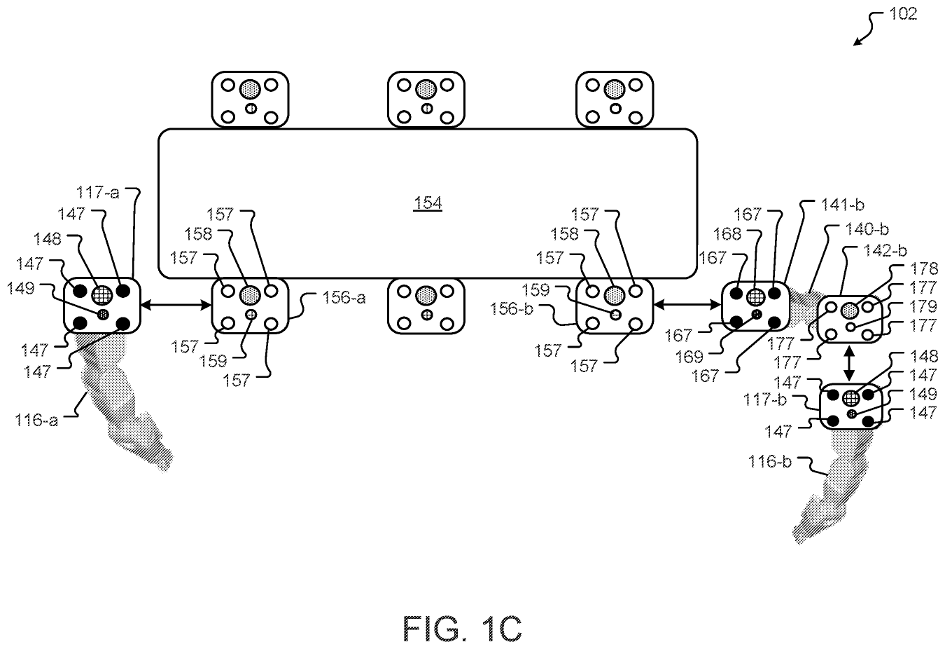

FIG. 1A illustrates an example of a system 100 that supports aspects of the present disclosure.

The system 100 includes a computing device 102, one or more imaging devices 112, a robot 114, a navigation system 118, a database 130, and/or a cloud network 134 (or other network). Systems according to other implementations of the present disclosure may include more or fewer components than the system 100. For example, the system 100 may omit and/or include additional instances of one or more components of the computing device 102, the imaging device(s) 112, the robot 114, navigation system 118, the database 130, and/or the cloud network 134. In an example, the system 100 may omit any instance of the computing device 102, the imaging device(s) 112, the robot 114, navigation system 118, the database 130, and/or the cloud network 134. The system 100 may support the implementation of one or more other aspects of one or more of the methods disclosed herein.

The computing device 102 includes a processor 104, a memory 106, a communication interface 108, and a user interface 110. Computing devices according to other implementations of the present disclosure may include more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging devices 112, the robot 114, the navigation system 118, the database 130, and/or the cloud network 134.

The memory 106 may be or include RAM, DRAM, SDRAM, other solid-state memory, any memory described herein, or any other tangible, non-transitory memory for storing computer-readable data and/or instructions. The memory 106 may store information or data associated with completing, for example, any step of the methods or process flows described herein, or of any other methods. The memory 106 may store, for example, instructions and/or machine learning models that support one or more functions of the imaging devices 112, the robot 114, and the navigation system 118. For instance, the memory 106 may store content (e.g., instructions and/or machine learning models) that, when executed by the processor 104, enable image processing 120, segmentation 122, transformation 124, registration 128, and/or an arm management engine 138. Such content, if provided as in instruction, may, in some implementations, be organized into one or more applications, modules, packages, layers, or engines.

Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging devices 112, the robot 114, the navigation system 118, the database 130, and/or the cloud network 134.

The computing device 102 may also include a communication interface 108. The communication interface 108 may be used for receiving data or other information from an external source (e.g., the imaging devices 112, the robot 114, the navigation system 118, the database 130, the cloud network 134, and/or any other system or component separate from the system 100), and/or for transmitting instructions (e.g., surgical commands associated with controlling the robotic arm 116 and/or an extension member coupled to the robotic arm 116), data (e.g., properties, pose information, etc. of a robotic arm 116 and/or extension member, image data, measurements, temperature information, etc.), or other information to an external system or device (e.g., another computing device 102, the imaging devices 112, the robot 114, the navigation system 118, the database 130, the cloud network 134, and/or any other system or component not part of the system 100). The communication interface 108 may include one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some implementations, the communication interface 108 may support communication between the device 102 and one or more other processors 104 or computing devices 102, whether to reduce the time needed to accomplish a computing-intensive task or for any other reason.

The computing device 102 may also include one or more user interfaces 110. The user interface 110 may be or include a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some implementations, the user interface 110 may support user modification (e.g., by a surgeon, medical personnel, a patient, etc.) of instructions to be executed by the processor 104 according to one or more implementations of the present disclosure, and/or to user modification or adjustment of a setting of other information displayed on the user interface 110 or corresponding thereto.

In some implementations, the computing device 102 may utilize a user interface 110 that is housed separately from one or more remaining components of the computing device 102. In some implementations, the user interface 110 may be located proximate one or more other components of the computing device 102, while in other implementations, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may include data corresponding to an anatomical feature of a patient, or to a portion thereof. The image data may be or include a preoperative image, an intraoperative image, a postoperative image, or an image taken independently of any surgical procedure. In some implementations, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or include, for example, an ultrasound scanner (which may include, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may include, for example, a transmitter, a receiver, a processor, and one or more antennae), or any other imaging device 112 suitable for obtaining images of an anatomical feature of a patient. The imaging device 112 may be contained entirely within a single housing, or may include a transmitter/emitter and a receiver/detector that are in separate housings or are otherwise physically separated.

In some implementations, the imaging device 112 may include more than one imaging device 112. For example, a first imaging device may provide first image data and/or a first image, and a second imaging device may provide second image data and/or a second image. In still other implementations, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or include, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some implementations, the robot 114 may be configured to hold and/or manipulate an anatomical element during or in connection with a surgical procedure. The robot 114 may include one or more robotic arms 116. In some implementations, the robotic arm 116 may include a first robotic arm and a second robotic arm, though the robot 114 may include more than two robotic arms. In some implementations, one or more of the robotic arms 116 may be used to hold and/or maneuver the imaging device 112. In implementations where the imaging device 112 includes two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may include one or more sensors that enable the processor 104 (or a processor of the robot 114) to determine a precise pose in space of the robotic arm (as well as any object or element held by or secured to the robotic arm).

In some implementations, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some implementations, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some implementations, the navigation system 118 may include one or more electromagnetic sensors. In various implementations, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some implementations, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The arm management engine 136 may support features described herein of identifying properties associated with a robotic arm 116, determining instruction operations and/or control parameters associated with controlling the robotic arm based on the identified properties, and controlling the robotic arm in association with a surgical procedure by transmitting surgical commands based the instruction operations and/or control parameters.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information.

The database 130 may be configured to provide any such information to the computing device 102 or to any other device of the system 100 or external to the system 100, whether directly or via the cloud network 134. In some implementations, the database 130 may be or include part of a hospital image storage system, such as a picture archiving and communication system (PACS), a health information system (HIS), and/or another system for collecting, storing, managing, and/or transmitting electronic medical records including image data.

In some aspects, the computing device 102 may communicate with a server(s) and/or a database (e.g., database 130) directly or indirectly over a communications network (e.g., the cloud network 134). The communications network may include any type of known communication medium or collection of communication media and may use any type of protocols to transport data between endpoints. The communications network may include wired communications technologies, wireless communications technologies, or any combination thereof.

Wired communications technologies may include, for example, Ethernet-based wired local area network (LAN) connections using physical transmission mediums (e.g., coaxial cable, copper cable/wire, fiber-optic cable, etc.). Wireless communications technologies may include, for example, cellular or cellular data connections and protocols (e.g., digital cellular, personal communications service (PCS), cellular digital packet data (CDPD), general packet radio service (GPRS), enhanced data rates for global system for mobile communications (GSM) evolution (EDGE), code division multiple access (CDMA), single-carrier radio transmission technology (1xRTT), evolution-data optimized (EVDO), high speed packet access (HSPA), universal mobile telecommunications service (UMTS), 3G, long term evolution (LTE), 4G, and/or 5G, etc.), Bluetooth®, Bluetooth® low energy, Wi-Fi, radio, satellite, infrared connections, and/or ZigBee® communication protocols.

The Internet is an example of the communications network that constitutes an Internet Protocol (IP) network consisting of multiple computers, computing networks, and other communication devices located in multiple locations, and components in the communications network (e.g., computers, computing networks, communication devices) may be connected through one or more telephone systems and other means. Other examples of the communications network may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communications network 120 may include of any combination of networks or network types. In some aspects, the communications network may include any combination of communication mediums such as coaxial cable, copper cable/wire, fiber-optic cable, or antennas for communicating data (e.g., transmitting/receiving data).

The computing device 102 may be connected to the cloud network 134 via the communication interface 108, using a wired connection, a wireless connection, or both. In some implementations, the computing device 102 may communicate with the database 130 and/or an external device (e.g., a computing device) via the cloud network 134.

The system 100 or similar systems may be used, for example, to carry out one or more aspects of any of the methods or process flows described herein. The system 100 or similar systems may also be used for other purposes.

FIG. 1B illustrates an example implementation 101 of the system 100 that supports aspects of the present disclosure. Aspects of the system 100 previously described with reference to FIG. 1A and descriptions of like elements are omitted for brevity.

Referring to the example implementation 101 in FIG. 1B, the system 100 may include a robot 114, robotic arms 116 (e.g., robotic arm 116-a through robotic arm 116-d, etc.), extension members 140 (e.g., extension member 140-b, extension member 140-c, etc.), a workstation 150, a support structure 154, and a support structure 155. In some examples, the workstation 150 may be integrated with the robot 114. In some other examples, the workstation 150 may be integrated with computing device 102 and/or the navigation system 118 described with reference to FIG. 1A.

The support structure 154 (e.g., a motorized or non-motorized operating table or platform, etc.) may be capable of supporting or holding a subject 152 during a procedure (e.g., a surgical procedure, medical imaging, etc.). The subject 152 may be a living subject (e.g., a human subject). It is understood, however, that the system 100 supports surgical procedures relative to any subject 152 using any combination of robotic arms 116 and extension members 140 described herein.

The support structure 155 may be, for example, mounted to a surface 158 of an environment in which the system 100 is implemented. The surface 158 may be a ceiling surface, a wall surface, a floor surface, or the like, and is not limited thereto. The environment may be, for example, an operating room and is not limited thereto.

The system 100 may support various purposes or procedures by one or more users (e.g., a surgeon, a medical technician, etc.) using any combination of robotic arms 116 and/or extension members 140. Aspects of the present disclosure support coupling of one or more robotic arms 116 and/or extension members 140 to any of the robot 114, the workstation 150, the support structure 154, and the support structure 155. For example, the robot 114, the support structure 154, and the support structure 155 may include respective mounting components 156 (e.g., mounting component 156-a through mounting component 156-d) to which the robotic arms 116 and extension members 140 may be coupled.

The term “coupled” may refer to a removable coupling between components (e.g., a robotic arm 116, an extension member 140, a mounting component 156, the support structure 154, the support structure 155, etc.). In an example, coupling a first component (e.g., robotic arm 116-b, etc.) to a second component (e.g., mounting component 156-a, extension member 140-b, etc.) may include releasably securing the first component to the second component.

The term “coupled” may refer to an electrical coupling and/or a mechanical coupling between the components. In an example, for a component (e.g., a robotic arm 116, an extension member 140, etc.) described herein as “coupled” to another component (e.g., another extension member 140, a mounting component 156, etc.), the coupling may include an electrical and/or mechanical coupling between the components. Electrical coupling may enable electrical signals to be shared between electrically coupled components. Mechanical coupling may enable one component to be physical supported and/or manipulated based on movements of another component mechanically coupled thereto.

The mounting components 156, ends 117 (e.g., end 117-a through end 117-d) of the robotic arms 116, ends 141 (e.g., end 141-b, end 141-c) of the extension members 140, and ends 142 (e.g., end 142-b, end 142-c) of the extension members 140 may include respective connection interfaces (also referred to herein as “coupling interfaces” or “interfaces”) supportive of plug-and-play interchangeability and flexibility of the robotic arms 116 and extension members 140. Examples of the connection interfaces are later described with reference to FIG. 1C.

According to example aspects of the present disclosure, the system 100 supports removably coupling an end 117 of a robotic arm 116 to a mounting component 156 in association with performing a surgical procedure or other procedure. For example, robotic arm 116-a may be coupled to mounting component 156-a of the support structure 154 via end 117-a of the robotic arm 116-a. In another example, robotic arm 116-d may be coupled to mounting component 156-d of the robot 114 (and workstation 150) via end 117-d of the robotic arm 116-d.

According to other example aspects of the present disclosure, the system 100 supports the use of an extension member 140 to increase the working range of a robotic arm 116. For example, the system 100 supports removably coupling an end 141 of an extension member 140 to a mounting component 156 and further, removably coupling an end 142 of the extension member 140 to an end 117 of a robotic arm 116 in association with performing a surgical procedure or other procedure. For example, end 141-b of the extension member 140-b may be coupled to mounting component 156-b of the support structure 154, and end 142-b of the extension member 140-b may be coupled to end 117-b of the robotic arm 116-b. In another example, end 141-c of the extension member 140-c may be coupled to mounting component 156-c of the support structure 155, and end 142-c of the extension member 140-c may be coupled to end 117-c of the robotic arm 116-c. The system 100 may support any combination of extension members 140 of different dimensions (e.g., different thickness, different lengths, etc.), different quantities of joints, and the like.

FIG. 1C illustrates an example implementation 102 of the system 100 that supports aspects of the present disclosure. Aspects of the system 100 previously described with reference to FIGS. 1A and 1B and descriptions of like elements are omitted for brevity.

Aspects of a connection interface of a robotic arm 116 supported by the present disclosure are described herein. In an example, referring to FIG. 1C, the connection interface of a robotic arm 116 may include one or more position-mounting holes 147 (e.g., four position-mounting holes 147), a power supply connector 148, and a communication connector 149. It is to be understood that the aspects of the connection interfaces of the robotic arms 116 (e.g., robotic arm 116-a, robotic arm 116-b) illustrated and described with reference to FIG. 1C may apply to any robotic arm 116 described herein.

Aspects of a connection interface of a mounting component 156 supported by the present disclosure are described herein. In an example, referring to FIG. 1C, the connection interface of a mounting component 156 may include one or more position-mounting holes 157 (e.g., four position-mounting holes 157), a power supply connector 158, a communication connector 159. It is to be understood that the aspects of the connection interfaces of the mounting components 156 (e.g., mounting component 156-a, mounting component 156-b) illustrated and described with reference to FIG. 1C may apply to any mounting component 156 described herein. For example, the aspects described with reference to FIG. 1C may apply to mounting component 156-a and mounting component 156-b of the support structure 154, mounting component 156-c of the support structure 155 of FIG. 1B, mounting component 156-d of the robot 114 of FIG. 1B, and the like.

Aspects of connection interfaces of an extension member 140 supported by the present disclosure (e.g., extension member 140-b and extension member 140-c) are described herein. In an example, referring to FIG. 1C, a connection interface at an end 141 of an extension member 140 may include one or more position-mounting holes 167 (e.g., four position-mounting holes 167), a power supply connector 168, and a communication connector 169. A connection interface at an end 142 of the extension member 140 may include one or more position-mounting holes 177 (e.g., four position-mounting holes 177), a power supply connector 178, and a communication connector 179. It is to be understood that the aspects of the connection interfaces of the extension member 140-b illustrated and described with reference to FIG. 1C may apply to any extension member 140 described herein.

The configurations and orientations of the connection interfaces of the robotic arms 116, the extension members 140, and the mounting components 156 as illustrated herein are examples, and it is to be understood that the configurations and orientations may be different from the examples. In some aspects, the position-mounting holes (e.g., position-mounting holes 147, position-mounting holes 157, position-mounting holes 167, position-mounting holes 177) may also be referred to as position-mounting interfaces.

In an example aspect, the connection interface of the robotic arm 116-a (and robotic arm 116-b) may match or complement the connection interfaces of the mounting component 156-a and the mounting component 156-b. For example, the configuration (e.g., arrangement, positioning, etc.), types, and sizes of the position-mounting holes 147, the power supply connector 148, and the communication connector 149 of the robotic arm 116-a may match or complement the configuration (e.g., arrangement, positioning, etc.), types, and sizes of the position-mounting holes 157, the power supply connector 158, and the communication connector 159 of the mounting components 156.

The connection interface of the robotic arm 116-a (and robotic arm 116-b) may match or complement the connection interface at end 142-b of the extension member 140-b. For example, the configuration (e.g., arrangement, positioning, etc.), types, and sizes of the position-mounting holes 147, the power supply connector 148, and the communication connector 149 of the robotic arm 116-a may match or complement the configuration (e.g., arrangement, positioning, etc.), types, and sizes of the position-mounting holes 177, the power supply connector 178, and the communication connector 179 of the extension member 140-b.

The connection interface at end 141-b of the extension member 140-b may match or complement the connection interfaces of the mounting component 156-a and the mounting component 156-b. For example, the configuration (e.g., arrangement, positioning, etc.), types, and sizes of the position-mounting holes 167, the power supply connector 168, and the communication connector 169 of the extension member 140-b may match or complement the configuration (e.g., arrangement, positioning, etc.), types, and sizes of the position-mounting holes 157, the power supply connector 158, and the communication connector 159 of the mounting components 156.

In an example, the gender (e.g., female or male) of the power supply connector 158, the communication connector 159, the power supply connector 178, and the communication connector 179 may be configured to complement the gender (e.g., male or female) of the power supply connector 148, the communication connector 149, the power supply connector 168 and the communication connector 169. In some other examples, aspects of the present disclosure support gender neutral (e.g., flat, non-recessed, non-protruding, etc.) implementations of any of the power supply connector 148, the communication connector 149, the power supply connector 158, the communication connector 159, the power supply connector 168, the communication connector 169, the power supply connector 178, and the communication connector 179. In some aspects, coupling between the connection interfaces described herein may be implemented with ferromagnetic connectors.

In some examples, the robotic arm 116-a may be coupled to the mounting component 156-a using pins (not illustrated) inserted at locations where the position-mounting holes 147 and the position-mounting holes 157 overlap. In another example, the extension member 140-b may be coupled to the mounting component 156-b using pins (not illustrated) inserted at locations where the position-mounting holes 167 and the position-mounting holes 157 overlap. The robotic arm 116-b may be coupled to the extension member 140-b using pins (not illustrated) inserted at locations where the position-mounting holes 147 and the position-mounting holes 177 overlap.

In some alternative aspects, the position-mounting holes 147 of the robotic arm 116-a may be replaced with pins, and the pins may be inserted into the position-mounting holes 157 of the mounting component 156-a in association with coupling the robotic arm 116-a to the mounting component 156-a. In another example, the position-mounting holes 147 of the robotic arm 116-b may be replaced with pins, and the pins may be inserted into the position-mounting holes 177 of the extension member 140-b in association with coupling the robotic arm 116-b to the extension member 140-b. In a further example, the position-mounting holes 167 of the extension member 140-b may be replaced with pins, and the pins may be inserted into the position-mounting holes 157 of the mounting component 156-b in association with coupling the extension member 140-b to the mounting component 156-b.

Such examples of compatibility between the connection interfaces (and associated position-mounting holes, power supply connectors, and communication connectors) may support the plug-and-play interchangeability and flexibility of the robotic arms 116 and extension members 140 as described herein.

As described herein, aspects of the plug-and-play features of the robotic arms 116 and the extension members 140 support implementations in which the workstation 150 may be compatible with multiple robotic arms 116 and extension members 140 for multiple types of surgical procedures (e.g., trauma surgery, spine surgery, joint surgery, etc.). The workstation 150 may identify and implement algorithms associated with performing a surgical procedure based on a robotic arm 116 and/or extension member 140. It is to be understood that features described herein as implemented by the system 100 may be implemented by a computing device 102, robot 114, navigation system 118, and/or workstation 150 described herein.

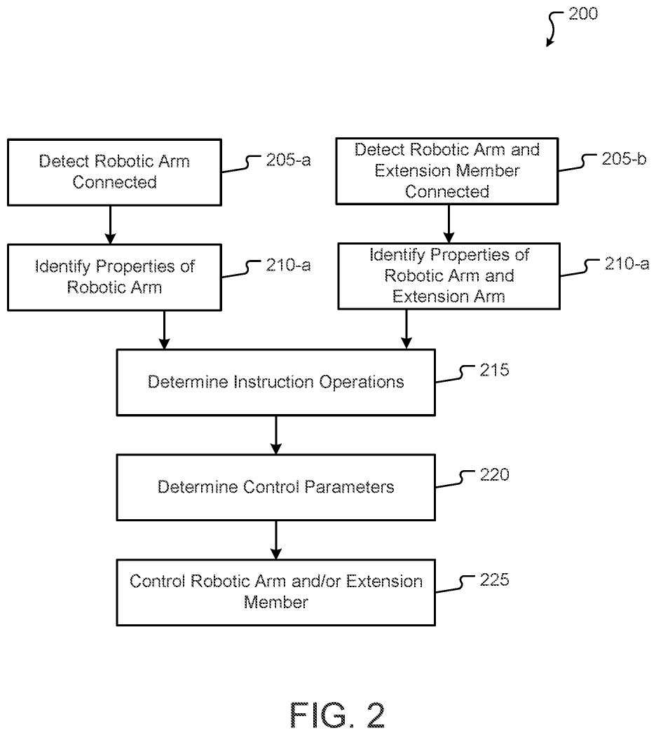

FIG. 2 illustrates an example of a process flow 200 that supports the plug-and-play features of robotic arms 116 and extension members 140 in accordance with aspects of the present disclosure. In some examples, process flow 200 may implement aspects of the system 100 described with reference to FIGS. 1A through 1C.

In the following description of the process flow 200, the operations may be performed in a different order than the order shown, or the operations may be performed in different orders or at different times. Certain operations may also be left out of the process flow 200, or other operations may be added to the process flow 200.

The example aspects of process flow 200 described herein with respect to controlling a robotic arm 116 and/or extension member 140 in association with a surgical procedure may be applied to any robotic arm 116 and extension member 140 supported by the system 100. It is to be understood that the example aspects are not limited to the robotic arms 116 (e.g., robotic arm 116-a, robotic arm 116-b) and extension members 140 (e.g., extension member 140-b) described with reference to the process flow 200.

At 205-a, the system 100 may detect that robotic arm 116-a has been connected (e.g., by a user) to the mounting component 156-a of a support structure such as the support structure 154. For example, the system 100 may detect that a connection (e.g., mechanical connection and/or electrical connection) has been established between a connection interface at end 117-a of the robotic arm 116-a and a connection interface of the mounting component 156-a.

In some cases, the system 100 may receive status data associated with the robotic arm 116-a indicating that the robotic arm 116-a is powered on and connected to the support structure 154. For example, the system 100 may electronically receive the status data from electronic circuitry integrated with the support structure 154. In another example, the system 100 may electronically receive the status data from the robotic arm 116-a.

In an example implementation, once coupled to the support structure 154, the robotic arm 116-a may receive power from the support structure 154 and enter a “powered on” state. In response to entering the “powered on” state, the robotic arm 116 may electronically transmit (e.g., wirelessly, or via the support structure 154) the status data of the robotic arm 116-a to the system 100.

At 205-b, the system 100 may detect that extension member 140-b and robotic arm 116-b have been connected (e.g., by a user) to the mounting component 156-b of the support structure 154. For example, the system 100 may detect that a connection (e.g., mechanical connection and/or electrical connection) has been established between a connection interface at end 141-b of the extension member 140-b and a connection interface of the mounting component 156-b. The system 100 may further detect that a connection has also been established between a connection interface at end 142-b of the extension member 140-b and a connection interface at end 117-b of the robotic arm 116-b.

In some cases, the system 100 may receive status data that the extension member 140-b is powered on and connected to the support structure 154. The status data may indicate that the robotic arm 116-b is powered on and connected to the support structure 154 via the extension member 140-b. For example, the system 100 may electronically receive the status data from electronic circuitry integrated with the support structure 154. In another example, the system 100 may electronically receive the status data from the extension member 140-b and/or the robotic arm 116-b.

In an example implementation, once coupled to the support structure 154, the extension member 140-b and the robotic arm 116-b may receive power from the support structure 154 and enter respective “powered on” states. In response to entering the “powered on” state, the extension member 140-b may electronically transmit (e.g., wirelessly, or via the support structure 154) the status data of the extension member 140-b and/or the robotic arm 116-b. In some examples, the robotic arm 116 may electronically transmit (e.g., wirelessly, or via the support structure 154) the status data of the robotic arm 116-b to the system 100.

At 210-a, the system 100 may identify properties associated with the robotic arm 116-a based on the established connection. For example, the system 100 may read data stored in the robotic arm 116-a (e.g., stored in a memory chip in the robotic arm 116-a), and the data may include an indication of the properties. In an example, the data may include identification information (e.g., a serial number, a model number, etc.) associated with the robotic arm 116-a, a hardware version associated with the robotic arm 116-a, a software version associated with the robotic arm 116-a, a type associated with the robotic arm 116-a, available functions of the robotic arm 116-a, and a range of motion of the robotic arm 116-a.

Additionally, or alternatively, the identification information may be visually detectable by the system 100. For example, the identification information may be physically integrated with the robotic arm 116-a (e.g., printed on the robotic arm 116-a, printed on a label attached to the robotic arm 116-a, etc.). The system 100 may visually (e.g., via an optical reading device, an optical sensor, a camera device, etc. included in the system 100) detect the identification information. Using the identification information, the system 100 may retrieve data from database 130 that indicates properties such as hardware version, software version, type, available functions, and range of motion of the robotic arm 116-a. Additionally, or alternatively, the system 100 may support receiving any of the identification information, hardware version, software version, type, available functions, and range of motion of the robotic arm 116-a via a user input at the user interface 110.

At 210-b, the system 100 may identify properties associated with the extension member 140-b and the robotic arm 116-b based on the established connection. For example, the system 100 may read data stored in the extension member 140-b (e.g., stored in a memory chip in the extension member 140-b) and/or data stored in the robotic arm 116-b (e.g., stored in a memory chip in the robotic arm 116-b), and the data may include an indication of the properties. In an example, the data stored in the extension member 140-b may include identification information (e.g., a serial number, a model number, etc.) associated with the extension member 140-b, a hardware version associated with the extension member 140-b, a software version associated with the extension member 140-b, a type associated with the extension member 140-b, available functions of the extension member 140-b, and a range of motion of the extension member 140-b. The data stored in the robotic arm 116-b may include identification information (e.g., a serial number, a model number, etc.), hardware version, software version, type, available functions, and range of motion of the robotic arm 116-b as described herein.

Additionally, or alternatively, the identification information of the extension member 140-b and/or the robotic arm 116-b may be visually detectable by the system 100. For example, the identification information of the extension member 140-b may be physically integrated with the extension member 140-b (e.g., printed on the extension member 140-b, printed on a label attached to the extension member 140-b, etc.). The identification information of the robotic arm 116-b may be physically integrated with the robotic arm 116-b (e.g., printed on the robotic arm 116-b, printed on a label attached to the robotic arm 116-b, etc.).

The system 100 may visually detect the identification information of the extension member 140-b and/or robotic arm 116-b. Using the identification information, the system 100 may retrieve data from database 130 that indicates respective properties of the extension member 140-b and the robotic arm 116-b, such as hardware version, software version, type, available functions, and range of motion. Additionally, or alternatively, the system 100 may support receiving any of the identification information, hardware version, software version, type, available functions, and range of motion of extension member 140-b and/or the robotic arm 116-b via a user input at the user interface 110.

At 215, the system 100 may determine instruction operations associated with controlling the robotic arm 116-a based the identification information and/or properties (e.g., hardware version, software version, type, available functions, range of motion, etc.) of the robotic arm 116-a. For example, at 215, the system 100 may identify capabilities of the robotic arm 116-a.

Additionally, or alternatively, at 215, the system 100 may determine instruction operations associated with controlling the extension member 140-b and the robotic arm 116-b based the respective identification information and/or properties (e.g., hardware version, software version, type, available functions, range of motion, etc.) of the extension member 140-b and the robotic arm 116-b. For example, at 215-b, the system 100 may identify capabilities of the extension member 140-b and the robotic arm 116-b.

At 220, the system 100 may determine or configure control parameters for controlling the robotic arm 116-a, based on the properties of the robotic arm 116-a. For example, at 220-a, the system 100 may determine algorithms for controlling the robotic arm 116-a, based on the capabilities determined at 215. The system 100 may determine or set the control parameters in association with a target objective (e.g., delivering therapy, performing a surgical incision, etc.) of the surgical procedure. In some aspects, the target objective may be set by the user via the workstation 150. An example control parameter includes target pose information of the robotic arm 116-a. Another example control parameter includes target pose information of an end effector of the robotic arm 116-a.

Additionally, or alternatively, at 220, the system 100 may determine or configure control parameters for controlling the extension member 140-b and the robotic arm 116-b, based on the respective properties of the extension member 140-b and the robotic arm 116-b. For example, at 220-b, the system 100 may determine algorithms for controlling the extension member 140-b and the robotic arm 116-b, based on the capabilities determined at 215-b. The system 100 may determine or set the control parameters in association with a target objective (e.g., delivering therapy, performing a surgical incision, etc.) of the surgical procedure. An example control parameter includes target pose information of the extension member 140-b and the robotic arm 116-b. Another example control parameter includes target pose information of an end effector of the robotic arm 116-b.

In some aspects, the system 100 may identify other data associated with a surgical procedure, and the system 100 may configure the control parameters (e.g., at 220-a and 220-b) based on the other data. Examples of the other data include characteristics (e.g., weight, height, pose information, etc.) of the subject 152, characteristics (e.g., dimensions, pose information, type, etc.) of a target anatomical element of the subject 152 in association with a surgical procedure, and the like. Additional examples of the other data include target coordinates associated with the surgical procedure, type of surgical procedure, and the like, and are not limited thereto. In an example, the system 100 may access the other data from a surgical plan described herein.

At 225, the system 100 may control the robotic arm 116-a in association with performing the surgical procedure. For example, the system 100 may control the robotic arm 116-a by providing surgical commands to the robotic arm 116-a that correspond to the instruction operations of 215-a and control parameters of 220-a.

Additionally, or alternatively, at 225, the system 100 may control the extension member 140-b and robotic arm 116-b in association with performing the surgical procedure. For example, the system 100 may control the extension member 140 and robotic arm 116-b by providing surgical commands to the extension member 140-b and robotic arm 116-b. The provided surgical commands may correspond to the instruction operations of 215-b and control parameters of 220-b.

The system 100 may monitor pose information of the robotic arm 116-a in association with the surgical procedure. For example, the robotic arm 116-a may electronically provide data to the system 100 indicative of the pose information. In another example, the system 100 may monitor pose information of the extension member 140-b and robotic arm 116-b in association with the surgical procedure. For example, the extension member 140-b and/or robotic arm 116-b (e.g., via the extension member 140-b) may electronically provide data to the system 100 indicative of the pose information. In some aspects, the system 100 may support tracking techniques (e.g., using cameras or other sensor(s) described with reference to navigation system 118) based on which the system 100 may monitor the pose information of the robotic arm 116-a, the extension member 140-b, and the robotic arm 116-b.

In some example implementations, the system 100 may provide alerts or recommendations (e.g., via the user interface 110, via the workstation 150, etc.) to the user indicating a recommended robotic arm 116, a recommended extension member 140, and/or a recommended installation location of the robotic arm 116 or extension member 140 for performing a surgical procedure. The system 100 may provide such alerts or recommendations preoperatively or intraoperatively.

For example, the system 100 may identify a target location (e.g., surgical site, an anatomical element, etc.) associated with the surgical procedure based on corresponding surgical plan stored in the database 130. In an example in which no robotic arms 116 are yet connected and activated, the system 100 may output a recommendation to install one or more robotic arms 116 (e.g., mount one or more robotic arms 116 and/or extension members 140 to a support structure) in association with performing the surgical procedure.

In another example in which one or more robotic arms 116 are connected and powered on, the system 100 may determine whether the properties (e.g., available functions, range of motion, etc.) of the robotic arm 116-a support performing the surgical procedure. For example, for the robotic arm 116-a coupled to mounting component 156-a, the system 100 may determine whether the properties (e.g., available functions, range of motion, etc.) of the robotic arm 116-a support performing the surgical procedure.

In an example case, if the available functions of the robotic arm 116-a do not support performing the surgical procedure, the system 100 may output a recommendation to replace the robotic arm 116-a with a robotic arm 116 of a different type. In another example, if the range of motion of the robotic arm 116-a does not support performing the surgical procedure (e.g., due to distance, trajectory, etc.), the system 100 may output a recommendation to add an extension member 140 between the robotic arm 116-a and the mounting component 156-a. Additionally, or alternatively, the system 100 may output a recommendation to mount the robotic arm 116-a at another location. For example, the system 100 may output a recommendation to mount the robotic arm 116-a (with or without an extension member 140) to the mounting component 156-c of support structure 155. In another example, the system 100 may output a recommendation to mount the robotic arm 116-a (with or without an extension member 140) to mounting component 156-d of the robot 114.

In another example case, the system 100 may determine from the surgical plan (or real-time measurement data) that the weight of the subject 152 in combination with the weight of the robotic arm 116-a and/or robotic arm 116-b may exceed the weight capacity of the support structure 154. The system 100 may output a recommendation to replace the robotic arm 116-a and/or the robotic arm 116-b with a comparable robotic arm 116 that weighs less. In another example, the system 100 may output a recommendation to relocate the robotic arm 116-a and/or the robotic arm 116-b to another support structure (e.g., support structure 155, a support structure of the robot 114, etc.). In some cases, the system 100 may support providing recommendations to place robotic arms 116 (and/or extension members 140) according to locations of different mounting components 156 of the support structure 154 to evenly distribute the combined weight of the robotic arms 116 and/or extension members 140.

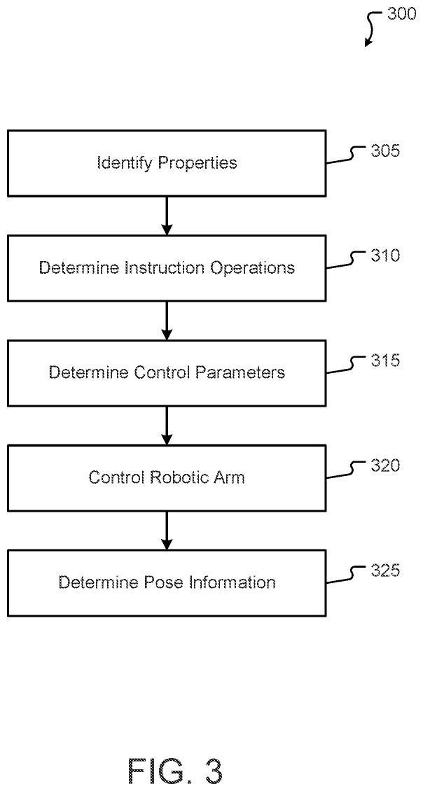

FIG. 3 illustrates an example of a process flow 300 in accordance with aspects of the present disclosure. In some examples, process flow 300 may implement aspects of the system 100 described with reference to FIGS. 1A through 1C.

In the following description of the process flow 300, the operations may be performed in a different order than the order shown, or the operations may be performed in different orders or at different times. Certain operations may also be left out of the process flow 300, or other operations may be added to the process flow 300.

The example aspects of process flow 300 described herein with respect to controlling a robotic arm 116 and/or extension member 140 in association with a surgical procedure may be applied to any robotic arm 116 and extension member 140 supported by the system 100. It is to be understood that the example aspects are not limited to the robotic arms 116 (e.g., robotic arm 116-a, robotic arm 116-b) and extension members 140 (e.g., extension member 140-b) described with reference to the process flow 300.

At 303, the process flow 300 may include detecting a removable coupling established between a robotic arm and coupling interface of a system. In some aspects, the removable coupling includes at least one of: a mechanical connection established between the robotic arm and the coupling interface; and an electrical connection established between the robotic arm and the coupling interface. In some aspects, the coupling interface is associated with one or more support structures of the system.

At 305, the process flow 300 may include identifying one or more properties associated with a robotic arm. In some aspects, the robotic arm is plug-and-play compatible with a coupling interface of the system. In some aspects, identifying the one or more properties associated with the robotic arm is in response to detecting the removable coupling. In some aspects, the process flow 300 may include retrieving the one or more properties associated with the robotic arm from a memory stored on the robotic arm.

In some aspects, the one or more properties associated with the robotic arm include at least one of: status information associated with the robotic arm; and identification information associated with the robotic arm. In some aspects, the one or more properties associated with the robotic arm include at least one of: a hardware version associated with the robotic arm; and a software version associated with the robotic arm. In some aspects, the one or more properties associated with the robotic arm include at least one of: a type associated with the robotic arm; one or more functions associated with the robotic arm; and a range of motion associated with the robotic arm.

At 310, the process flow 300 may include determining one or more instruction operations associated with controlling the robotic arm based on the one or more properties.

In some aspects, determining the one or more instruction operations is based on a target objective associated with performing the procedure.

At 315, the process flow 300 may include determining one or more control parameters associated with controlling the robotic arm based on the one or more properties associated with the robotic arm.

In some aspects, the one or more control parameters include at least one of: target pose information of the robotic arm; and second target pose information of an end effector of the robotic arm. In some aspects, determining the one or more instruction operations, determining the one or more control parameters, or both is based on at least one of: one or more characteristics of a subject associated with the surgical procedure; and one or more characteristics of an anatomical element of the subject in association with the surgical procedure.

At 320, the process flow 300 may include controlling the robotic arm in association with a procedure by transmitting one or more commands based on the one or more instruction operations. In some aspects, controlling the robotic arm includes by transmitting the one or more surgical commands based on the one or more control parameters.

At 325, the process flow 300 may include determining pose information of the robotic arm, pose information of an extension member removably coupled to the robotic arm, or both in response to at least one of: receiving data from the robotic arm; and receiving second data from an extension member, wherein the extension member is removably coupled to the coupling interface.

In some aspects (not illustrated), the process flow 300 includes: identifying one or more second properties associated with an extension member, wherein the extension member is plug-and-play compatible with the coupling interface and the robotic arm; and the extension member is removably coupled to the coupling interface and removably coupled to the robotic arm; determining one or more second control parameters associated with controlling the extension member and the robotic arm based on identifying the one or more second properties; and controlling the extension member and the robotic arm in association with the procedure by transmitting the one or more surgical commands based on the one or more second control parameters.

In some aspects, the one or more second properties include at least one of: a type associated with the extension member; one or more functions associated with the extension member; and a range of motion associated with the extension member.

The process flows 200 and 300 described herein (and/or one or more operations thereof) may be carried out or otherwise performed, for example, by at least one processor. The at least one processor may be the same as or similar to the processor(s) 104 of the computing device 102 described above. The at least one processor may be part of a robot (such as a robot 114), part of a navigation system (such as a navigation system 118), or part of a workstation 150. A processor other than any processor described herein may also be used to execute the process flows described herein. The at least one processor may perform operations of the process flows by executing elements stored in a memory such as the memory 106. The elements stored in memory and executed by the processor may cause the processor to execute one or more operations of a function as shown in the process flows. One or more portions of the process flows may be performed by the processor executing any of the contents of memory, such as arm management engine 136, image processing 120, a segmentation 122, a transformation 124, and/or a registration 128.

As noted above, the present disclosure encompasses methods with fewer than all of the operations identified in FIGS. 2 and 3 (and the corresponding descriptions of the process flows 200 and 300), as well as methods that include additional operations beyond those identified in FIGS. 2 and 3. The present disclosure also encompasses methods that include one or more steps from one method described herein, and one or more steps from another method described herein. Any correlation described herein may be or include a registration or any other correlation.

The foregoing is not intended to limit the disclosure to the form or forms disclosed herein. In the foregoing Detailed Description, for example, various features of the disclosure are grouped together in one or more aspects, implementations, and/or configurations for the purpose of streamlining the disclosure. The features of the aspects, implementations, and/or configurations of the disclosure may be combined in alternate aspects, implementations, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, implementation, and/or configuration. Thus, the following claims are hereby incorporated into this Detailed Description, with each claim standing on its own as a separate preferred implementation of the disclosure.

Moreover, though the foregoing has included description of one or more aspects, implementations, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, implementations, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Example aspects of the present disclosure include:

A system including: a processor; and a memory storing instructions thereon that, when executed by the processor, cause the processor to: identify one or more properties associated with a robotic arm, wherein the robotic arm is plug-and-play compatible with a coupling interface of the system; determine one or more instruction operations associated with controlling the robotic arm based on the one or more properties; and control the robotic arm in association with a surgical procedure by transmitting one or more surgical commands based on the one or more instruction operations.

Any of the aspects herein, wherein the instructions are further executable by the processor to: detect a removable coupling established between the robotic arm and the coupling interface, wherein the removable coupling includes at least one of: a mechanical connection established between the robotic arm and the coupling interface; and an electrical connection established between the robotic arm and the coupling interface, wherein identifying the one or more properties associated with the robotic arm is in response to detecting the removable coupling.

Any of the aspects herein, wherein the instructions are further executable by the processor to: retrieve the one or more properties associated with the robotic arm from a memory stored on the robotic arm.

Any of the aspects herein, wherein the one or more properties associated with the robotic arm include at least one of: status information associated with the robotic arm; and identification information associated with the robotic arm.

Any of the aspects herein, wherein the one or more properties associated with the robotic arm include at least one of: a hardware version associated with the robotic arm; and a software version associated with the robotic arm.

Any of the aspects herein, wherein the one or more properties associated with the robotic arm include at least one of: a type associated with the robotic arm; one or more functions associated with the robotic arm; and a range of motion associated with the robotic arm.

Any of the aspects herein, further including: determine one or more control parameters associated with controlling the robotic arm based on the one or more properties associated with the robotic arm, wherein the one or more control parameters include at least one of target pose information of the robotic arm and second target pose information of an end effector of the robotic arm, wherein controlling the robotic arm includes by transmitting the one or more surgical commands based on the one or more control parameters.

Any of the aspects herein, wherein determining the one or more instruction operations, determining the one or more control parameters, or both is based on at least one of: one or more characteristics of a subject associated with the surgical procedure; and one or more characteristics of an anatomical element of the subject in association with the surgical procedure.

Any of the aspects herein, wherein the instructions are further executable by the processor to: identify one or more second properties associated with an extension member, wherein: the extension member is plug-and-play compatible with the coupling interface and the robotic arm; and the extension member is removably coupled to the coupling interface and removably coupled to the robotic arm; determine one or more second control parameters associated with controlling the extension member and the robotic arm based on identifying the one or more second properties; and control the extension member and the robotic arm in association with the procedure by transmitting the one or more surgical commands based on the one or more second control parameters.

Any of the aspects herein, wherein the one or more second properties include at least one of: a type associated with the extension member; one or more functions associated with the extension member; and a range of motion associated with the extension member.

Any of the aspects herein, wherein determining the one or more instruction operations is based on a target objective associated with performing the procedure.

Any of the aspects herein, wherein the instructions are further executable by the processor to: determine pose information of the robotic arm, pose information of an extension member removably coupled to the robotic arm, or both in response to at least one of: receiving data from the robotic arm; and receiving second data from an extension member, wherein the extension member is removably coupled to the coupling interface.

Any of the aspects herein, wherein the coupling interface is associated with one or more support structures of the system.

A system including: a robotic arm; and robot management circuitry that manages control of the robotic arm by: identifying one or more properties associated with the robotic arm, wherein the robotic arm is plug-and-play compatible with a mounting component of the system; determining one or more instruction operations associated with controlling the robotic arm based on identifying the one or more properties; and controlling the robotic arm in association with a surgical procedure by transmitting one or more surgical commands based on the one or more instruction operations.

Any of the aspects herein, wherein the robot management circuitry is to: detect a removable coupling established between the robotic arm and the coupling interface, wherein the removable coupling includes at least one of: a mechanical connection established between the robotic arm and the coupling interface; and an electrical connection established between the robotic arm and the coupling interface, wherein identifying the one or more properties associated with the robotic arm is in response to detecting the removable coupling

Any of the aspects herein, wherein the robot management circuitry further manages control of the robotic arm by: determining one or more control parameters associated with controlling the robotic arm based on identifying the one or more properties associated with the robotic arm, wherein controlling the robotic arm includes by transmitting the one or more surgical commands based on identifying the one or more control parameters.

Any of the aspects herein, wherein the robot management circuitry further manages control of the robotic arm by: identifying one or more second properties associated with an extension member, wherein: the extension member is plug-and-play compatible with the coupling interface and the robotic arm; and the extension member is removably coupled to the coupling interface of and removably coupled to the robotic arm; determining one or more second control parameters associated with controlling the extension member and the robotic arm based on identifying the one or more second properties; and controlling the extension member and the robotic arm in association with the procedure by transmitting the one or more surgical commands based on the one or more second control parameters.

Any of the aspects herein, further including a support structure including the mounting component, wherein: the mounting component includes one or more position-mounting interfaces, one or more power interfaces, and one or more data communication interfaces; and the robotic arm is plug-and-play compatible with the mounting component based on one or more second position-mounting interfaces, one or more second power interfaces, and one or more second data communication interfaces of the robotic arm.

Any of the aspects herein, further including an extension member removably coupled to the robotic arm and to the mounting component, wherein: a first end of the extension member is plug-and-play compatible with the mounting component based on one or more position-mounting interfaces, one or more power interfaces, and one or more data communication interfaces of the extension member; and a second end of the extension member is plug-and-play compatible with the robotic arm based on one or more second position-mounting interfaces, one or more second power interfaces, and one or more second data communication interfaces of the extension member.

A method including: electronically receiving status information associated with a robotic arm of a system, wherein the status information includes an indication that the robotic arm is in an active state, wherein the robotic arm is plug-and-play compatible with a coupling interface of the system; and determining, in response to electronically receiving the status information and based on one or more properties associated with the robotic arm, one or more instruction operations associated with controlling the robotic arm; and controlling the robotic arm in association with a surgical procedure by electronically communicating one or more surgical commands corresponding to the one or more instruction operations to the robotic arm.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A system comprising:
a processor; and
a memory storing instructions thereon that, when executed by the processor, cause the processor to:
identify one or more properties associated with a robotic arm, wherein the robotic arm is plug-and-play compatible with a coupling interface of the system;
determine one or more instruction operations associated with controlling the robotic arm based at least in part on the one or more properties; and
control the robotic arm in association with a surgical procedure by transmitting one or more surgical commands based at least in part on the one or more instruction operations.

2. The system of claim 1, wherein the instructions are further executable by the processor to:
detect a removable coupling established between the robotic arm and the coupling interface, wherein the removable coupling comprises at least one of:
a mechanical connection established between the robotic arm and the coupling interface; and
an electrical connection established between the robotic arm and the coupling interface,
wherein identifying the one or more properties associated with the robotic arm is in response to detecting the removable coupling.

3. The system of claim 1, wherein the instructions are further executable by the processor to:
retrieve the one or more properties associated with the robotic arm from a memory stored on the robotic arm.

4. The system of claim 1, wherein the one or more properties associated with the robotic arm comprise at least one of:
status information associated with the robotic arm; and
identification information associated with the robotic arm.

5. The system of claim 1, wherein the one or more properties associated with the robotic arm comprise at least one of:
a hardware version associated with the robotic arm; and
a software version associated with the robotic arm.

6. The system of claim 1, wherein the one or more properties associated with the robotic arm comprise at least one of:
a type associated with the robotic arm;
one or more functions associated with the robotic arm; and
a range of motion associated with the robotic arm.

7. The system of claim 1, further comprising:
determine one or more control parameters associated with controlling the robotic arm based at least in part on the one or more properties associated with the robotic arm, wherein the one or more control parameters comprise at least one of target pose information of the robotic arm and second target pose information of an end effector of the robotic arm,
wherein controlling the robotic arm comprises by transmitting the one or more surgical commands based at least in part on the one or more control parameters.

8. The system of claim 7, wherein determining the one or more instruction operations, determining the one or more control parameters, or both is based on at least one of:
one or more characteristics of a subject associated with the surgical procedure; and
one or more characteristics of an anatomical element of the subject in association with the surgical procedure.

9. The system of claim 1, wherein the instructions are further executable by the processor to:
identify one or more second properties associated with an extension member, wherein:
the extension member is plug-and-play compatible with the coupling interface and the robotic arm; and
the extension member is removably coupled to the coupling interface and removably coupled to the robotic arm;
determine one or more second control parameters associated with controlling the extension member and the robotic arm based at least in part on identifying the one or more second properties; and
control the extension member and the robotic arm in association with the procedure by transmitting the one or more surgical commands based at least in part on the one or more second control parameters.

10. The system of claim 9, wherein the one or more second properties comprise at least one of:
a type associated with the extension member;
one or more functions associated with the extension member; and
a range of motion associated with the extension member.

11. The system of claim 1, wherein determining the one or more instruction operations is based at least in part on a target objective associated with performing the procedure.

12. The system of claim 1, wherein the instructions are further executable by the processor to:
determine pose information of the robotic arm, pose information of an extension member removably coupled to the robotic arm, or both in response to at least one of:
receiving data from the robotic arm; and
receiving second data from an extension member, wherein the extension member is removably coupled to the coupling interface.

13. The system of claim 1, wherein the coupling interface is associated with one or more support structures of the system.

14. A system comprising:
a robotic arm; and
robot management circuitry that manages control of the robotic arm by:
identifying one or more properties associated with the robotic arm, wherein the robotic arm is plug-and-play compatible with a mounting component of the system;
determining one or more instruction operations associated with controlling the robotic arm based at least in part on identifying the one or more properties; and
controlling the robotic arm in association with a surgical procedure by transmitting one or more surgical commands based at least in part on the one or more instruction operations.

15. The system of claim 14, wherein the robot management circuitry is to:
detect a removable coupling established between the robotic arm and the coupling interface, wherein the removable coupling comprises at least one of:
a mechanical connection established between the robotic arm and the coupling interface; and
an electrical connection established between the robotic arm and the coupling interface,
wherein identifying the one or more properties associated with the robotic arm is in response to detecting the removable coupling

16. The system of claim 14, wherein the robot management circuitry further manages control of the robotic arm by:
determining one or more control parameters associated with controlling the robotic arm based at least in part on identifying the one or more properties associated with the robotic arm,
wherein controlling the robotic arm comprises by transmitting the one or more surgical commands based at least in part on identifying the one or more control parameters.

17. The system of claim 14, wherein the robot management circuitry further manages control of the robotic arm by:
identifying one or more second properties associated with an extension member, wherein:
the extension member is plug-and-play compatible with the coupling interface and the robotic arm; and
the extension member is removably coupled to the coupling interface of and removably coupled to the robotic arm;
determining one or more second control parameters associated with controlling the extension member and the robotic arm based at least in part on identifying the one or more second properties; and
controlling the extension member and the robotic arm in association with the procedure by transmitting the one or more surgical commands based at least in part on the one or more second control parameters.

18. The system of claim 14, further comprising a support structure comprising the mounting component, wherein:
the mounting component comprises one or more position-mounting interfaces, one or more power interfaces, and one or more data communication interfaces; and
the robotic arm is plug-and-play compatible with the mounting component based on one or more second position-mounting interfaces, one or more second power interfaces, and one or more second data communication interfaces of the robotic arm.

19. The system of claim 14, further comprising an extension member removably coupled to the robotic arm and to the mounting component, wherein:
a first end of the extension member is plug-and-play compatible with the mounting component based on one or more position-mounting interfaces, one or more power interfaces, and one or more data communication interfaces of the extension member; and
a second end of the extension member is plug-and-play compatible with the robotic arm based on one or more second position-mounting interfaces, one or more second power interfaces, and one or more second data communication interfaces of the extension member.

20. A method comprising:
electronically receiving status information associated with a robotic arm of a system, wherein the status information comprises an indication that the robotic arm is in an active state, wherein the robotic arm is plug-and-play compatible with a coupling interface of the system; and
determining, in response to electronically receiving the status information and based at least in part on one or more properties associated with the robotic arm, one or more instruction operations associated with controlling the robotic arm; and
controlling the robotic arm in association with a surgical procedure by electronically communicating one or more surgical commands corresponding to the one or more instruction operations to the robotic arm.
